(19) United States
(12) Patent Application Publication
(10) Pub. No.: US 2026/0325166 A1
(43) Pub. Date: Oct. 22, 2026

(54) MEDICAL DEVICE WITH ROTATIONALLY VARIANT MAGNETIC ELEMENT AND SYSTEM FOR USING SAME

(71) Applicant: Rivanna Medical, Inc., Charlottesville, VA (US)

(72) Inventors: Frank William Mauldin, JR., Troy, VA (US); Adam Dixon, Charlottesville, VA (US); Kevin Owen, Stuart's Draft, VA (US)

(21) Appl. No.: 19/419,724

(22) Filed: Dec. 15, 2025

Related U.S. Application Data
(63) Continuation of application No. 19/247,487, filed on Jun. 24, 2025.
(60) Provisional application No. 63/791,294, filed on Apr. 18, 2025.

(51) Int. Cl. A61B 17/34; A61B 17/00; A61B 34/00; A61B 34/10; A61B 34/20; A61B 90/00

(52) U.S. Cl. CPC A61B 17/3403; A61B 34/10; A61B 34/20; A61B 34/25; A61B 90/08; A61B 2017/00039; A61B 2017/00106; A61B 2017/00154; A61B 2017/00199; A61B 2017/00734; A61B 2017/3405; A61B 2034/102; A61B 2034/107; A61B 2034/2051; A61B 2090/0807

(57) ABSTRACT

A device and related system for tracking a needle during percutaneous medical procedures using a magnetic element associated with the needle, the magnetic element producing an asymmetric magnetic field wherein spatial distribution changes measurably as the magnetic element is rotated.

### CROSS-REFERENCE TO RELATED APPLICATIONS

The present application claims priority to and the benefit of the filing date of U.S. patent application Ser. No. 19/247,487, filed Jun. 24, 2025, which claims priority to and the benefit of the filing date of U.S. Provisional Application No. 63/791,294, filed Apr. 18, 2025. The disclosures of those applications are hereby incorporated by reference herein in their entireties.

### STATEMENT REGARDING FEDERALLY SPONSORED RESEARCH

This invention was made with government support under Grant No. R44AR083337 awarded by the National Institutes of Health (NIH) National Institute of Arthritis and Musculoskeletal and Skin Diseases (NIAMS). The government has certain rights in the invention.

### BACKGROUND

### Field of the Invention

The present invention is related to medical interventional procedure guidance and tracking devices, as well as systems for using the tracking devices, such as for ultrasound imaging purposes, and more specifically to a magnetically trackable needle or catheter assembly used in image-guided medical procedures.

The present invention relates generally to medical devices and interventional instruments used in image-guided or computer-assisted procedures. More particularly, the invention pertains to needle or catheter assemblies incorporating one or more magnetic elements that can be detected, localized, and tracked, by external sensing systems during insertion and/or navigation within biological tissue or anatomy. Such devices and instruments can be used, according to the present invention, in conjunction with real-time imaging modalities, such as ultrasound, computed tomography, fluoroscopy, or magnetic resonance imaging, to provide feedback regarding the instrument's (e.g., needle or catheter shaft) position, orientation, and/or trajectory relative to an anatomical region.

In embodiments, the invention is directed to needle or catheter assemblies configured for integration with magnetic-field sensing arrays, electromagnetic tracking modules, and/or hybrid sensor platforms that combine magnetic and imaging data. The disclosed needle or catheter assemblies in conjunction with the current invention can create a system, such as for ultrasound imaging, that can enable continuous determination of both three-dimensional position and angular orientation of the needle or catheter shaft without requiring optical line of sight, allowing accurate guidance and tracking in environments where the instrument is partially or fully obscured from view.

### Description of Related Art

Magnetic tracking of needles is generally a static state of the art, and most proposed use cases do not allow for more robust and flexible user-control of the devices and system applications.

Conventional magnetic-tracking needle assemblies typically incorporate a permanent magnet aligned coaxially with the longitudinal axis of the needle shaft. The magnetic field produced by such a configuration extends along the cannula's longitudinal axis, and its geometry is straightforward to model and track. For this reason, coaxially magnetized designs have become the default approach in commercial and academic implementations of magnetically guided needle systems.

However, coaxial magnetization imposes significant functional and operational constraints. In particular, the magnetic field of an axially aligned magnet is rotationally symmetric about the cannula's longitudinal axis. This rotational symmetry yields field measurements that remain substantially invariant under rotation of the needle about its axis. As a result, a sensor array placed external to the patient cannot distinguish, for example, the roll orientation of the needle or detect whether the clinician has rotated the needle or magnet during insertion. Roll angle, however, directly determines the orientation of the needle bevel and thus influences the direction of tissue deflection, fluid dispersion, or the trajectory of a flexible stylet introduced through the lumen.

Because the magnetic field of a coaxially aligned magnet provides no unique rotational signature, sensor systems are effectively blind to angular motion about the longitudinal axis of the needle cannula. Orientation estimates derived from such symmetric fields are ambiguous and may drift as the system attempts to reconcile sensor noise with an under-constrained magnetic model. In practice, the inability to resolve roll orientation limits the accuracy of three-dimensional tracking and constrains the range of feedback that can be displayed to the user.

Moreover, a coaxially aligned magnet's symmetry demands tight manufacturing tolerances. Small deviations in angular placement or magnetization direction translate into systematic calibration errors that propagate through the sensor model. Since the magnet's field direction is fixed, any misalignment introduced during manufacture or at any other time persists throughout the product's lifetime. To mitigate this, conventional devices must often be assembled using jigs or fixtures that ensure precise, fixed, and known angular alignment of the magnetic axis relative to the hub or longitudinal axis of the cannula. Each assembly may require individual verification or recalibration to ensure consistency across production lots, increasing cost and reducing interchangeability of disposable components.

An additional limitation arises during magnetic calibration. Environmental magnetic fields, such as the Earth's field or local ferromagnetic bias from surrounding equipment, introduce offsets that must be compensated by the tracking system. In fixed-magnet assemblies, these biases cannot easily be separated from the magnet's intrinsic field, making calibration dependent on external reference frames or complex filtering algorithms. Because the magnet has a rotationally symmetric magnetic field, the system lacks a natural means of distinguishing static background magnetic field components from the signal of interest.

Accordingly, an improvement is needed in the relevant industry permitting more accurate, adjustable, efficacious, and/or safe alternatives to options currently available.

### SUMMARY OF THE INVENTION

The present invention includes a magnetic element, wherein the magnetic axis is intentionally non-coaxial with the longitudinal axis of the needle or catheter shaft. Herein, when the term needle (or variations thereof, such as needle cannula) is used, it also refers to a catheter shaft unless stated otherwise). In various embodiments, the magnetic axis may be oblique, diagonal skewed, orthogonal, and/or it can be transverse, relative to the longitudinal axis of the needle, producing an asymmetric magnetic field wherein spatial distribution changes measurably as the magnetic element is rotated, such as by a user during a medical procedure requiring the needle to be inserted into a patient, a patient anatomy, and/or a patient anatomical region (e.g., such as rotated by a doctor or a medical practitioner performing the medical procedure, by way of example only). This field asymmetry can provide unique angular features that magnetic-sensor arrays can detect, enabling a system (such as an ultrasound imaging system using the magnetic element and related device(s)) to resolve both position and rotational orientation, including roll angle, without relying on, in aspects, any fixed angular reference or pre-oriented magnetic axis position established at assembly.

According to the current invention, because the magnetic field pattern itself can encode the magnet's orientation, fixed alignment of the magnetic axis relative to the longitudinal axis of the needle cannula during manufacturing, for example, is no longer required. According to the current invention, variation in the alignment of the magnetic axis relative to the needle cannula can be characterized dynamically when introduced into the sensing environment. In aspects, the sensing system can measure the three-dimensional field vector, determine the magnetic axis orientation, and thereafter compute the position and/or trajectory of the needle with full six-degree-of-freedom accuracy. Variations in magnetic axis rotation or tilt can be automatically accommodated by the model, simplifying assembly processes and enabling interchangeable disposables.

The medical device (and associated system(s)) further provides for intentional rotational freedom of the magnetic element and its magnetization axis relative to the hub or needle cannula. The magnetic element may rotate freely or under controlled torque relative to the hub, allowing deliberate manipulation by the user or automatic calibration by the system. When the magnetic element is rotated, for example, its asymmetric field varies predictably, allowing an associated processor to measure changes in the detected field and to remove constant or slowly varying environmental magnetic contributions. This capability enables on-demand calibration routines that effectively or substantially null static bias without external equipment, in aspects.

User-controlled rotation also introduces a novel interaction modality that uses magnetic gestures as inputs to the sensor system. The user may intentionally rotate the magnetic element to send command inputs recognized by the tracking system, such as initiating data capture, marking procedural events, or toggling display modes, without contacting any non-sterile interface, in aspects. In this way, the magnetic medical device or elements thereof function not only as a passive tracking target, but also as an active communication interface integrated within the sterile field.

In embodiments, collectively the combination of non-coaxial magnetic axis geometry and rotational variability transforms the role of the magnet from a static reference into an actively measurable and controllable component of the medical device and tracking system. Thus, the resulting needles and/or catheter shafts of the device and system exhibit one or more of: greater calibration robustness, improved angular accuracy, enhanced manufacturing tolerance, and expanded functionality, establishing novel use modes and applications for magnetically trackable interventional devices, such as for use with ultrasound systems.

More specifically, applications include, but are not limited to, regional anesthesia, targeted drug delivery, fine-needle aspiration, tissue biopsy, vascular access, catheter and/or catheter shaft placement, and other invasive (or minimally invasive) diagnostic or therapeutic procedures in which the user benefits from real-time feedback on needle position and orientation relative to a patient's anatomy.

In embodiments, the medical device operates as a component within a multimodal imaging and navigation system that fuses magnetic field measurements with ultrasound, fluoroscopic, or tomographic data (and/or images/videos). This integration can provide the user with synchronized imaging and tracking information, supporting safer and more efficient procedural workflows.

The invention is also applicable to robotic and computer-assisted interventions in which a mechanical manipulator or positioning device inserts or steers a magnetically trackable needle. In such embodiments, the non-coaxial magnetic axis provides a continuously observable angular reference for the robot controller, permitting closed-loop correction of pitch, yaw, and roll deviations. Because the magnet's orientation can be detected without visual cues, robotic systems may operate in environments with limited line of sight or opaque media.

The invention is also applicable to medical instruments that penetrate the epidermis during percutaneous procedures other than needles, for example catheter shafts, trocars, and/or biopsy punches.

Beyond clinical medicine, the disclosed devices and related systems may be adapted for veterinary procedures, preclinical research, and industrial or laboratory systems that call for accurate localization of penetrating probes within nontransparent media. According to the current invention, detecting a rotatable, non-coaxial magnetic element to infer position and orientation may further be applied to instruments such as biopsy guns, trocars, guidewires, and steerable catheters (including catheter shafts).

While the examples above and herein illustrate certain medical and surgical applications, the scope of the invention is not limited to any particular field of use. Any procedure or device in which spatial localization, orientation tracking, or user-controlled calibration of an invasive or semi-invasive instrument is advantageous, may benefit from the medical device, structures, materials, magnetic configurations, systems, and ultrasound systems, described herein.

### BRIEF DESCRIPTION OF THE DRAWINGS

The accompanying drawings illustrate certain aspects of some of the embodiments of the present invention and should not be used to limit or define the invention. Together with the written description the drawings serve to explain certain principles of the invention. For a fuller understanding of the nature and advantages of the present technology, reference is made to the following detailed description of preferred embodiments and in connection with the accompanying drawings, in which:

FIG. 1 depicts an exemplary medical device that incorporates a ring magnet, according to embodiments described herein.

FIG. 2 depicts an exemplary medical device that incorporates a solid cylindrical magnet, according to embodiments described herein.

FIG. 3 depicts an exemplary ultrasound imaging system with ultrasound probe that integrates the magnetic sensor device (e.g., including the magnetic element and/or magnetic sensor array) for operation with a magnetically trackable medical instrument, such as a needle or catheter shaft, according to embodiments described herein.

FIG. 4 depicts an exemplary magnetically trackable medical device in operation with a medical ultrasound imaging probe, according to embodiments described herein.

FIG. 5 depicts an exemplary magnetic field sensor array integrated within a medical ultrasound probe and an exemplary magnetically trackable medical device, according to embodiments described herein.

FIG. 6 depicts an exemplary magnetically trackable medical device that incorporates a ring magnet that has undergone a rotation of its magnetic axis, according to embodiments described herein.

FIG. 7 depicts an exemplary user-interface workflow based on magnetic gestures resulting from different rotational states of a magnetically trackable medical instrument, according to embodiments described herein.

FIG. 8 shows a flowchart regarding how to use the medical device for ultrasound procedures or other applications, according to embodiments described herein.

FIG. 9 depicts an exemplary medical device, according to embodiments described herein.

FIG. 10 depicts an exemplary system using an exemplary medical device that incorporates a ring magnet, according to embodiments described herein.

### DETAILED DESCRIPTION OF VARIOUS EMBODIMENTS OF THE INVENTION

Various embodiments of the present invention relate to a medical device configured for magnetic tracking and orientation sensing during percutaneous medical procedures. In aspects, the device includes a hub and a needle or catheter shaft cannula extending distally from the hub along a longitudinal axis, with a magnetic element attached to, positioned within, located near, located on, or located next to the hub. The magnetic element defines a magnetic axis that is non-coaxial with the longitudinal axis of the cannula and can be mounted for rotational movement such that the magnetic axis is free to change orientation relative to the cannula (such as the longitudinal axis of the cannula). Rotation of the magnetic element produces a detectable variation in the surrounding magnetic field, enabling one or more external magnetic sensors (such as a sensor array) to sense and/or determine the element's angular position and, by extension, in aspects, the orientation of the medical instrument. The non-coaxial arrangement provides an asymmetric magnetic field that can be characterized by the sensing system (e.g., magnetic sensor array), allowing both position and rotational information to be derived from the measured field. In embodiments, the magnetic element is a cylindrical or ring magnet supported in a rotational sleeve or bearing, and may include visual or tactile fiducials corresponding to the orientation of the magnetic axis, such as, in aspects, to facilitate manual calibration or alignment by a user. The rotatable configuration can also enable in situ calibration routines that measure and optionally cancel static magnetic bias fields without requiring, in aspects, disassembly or external alignment tools, thereby improving tracking accuracy and simplifying manufacturing.

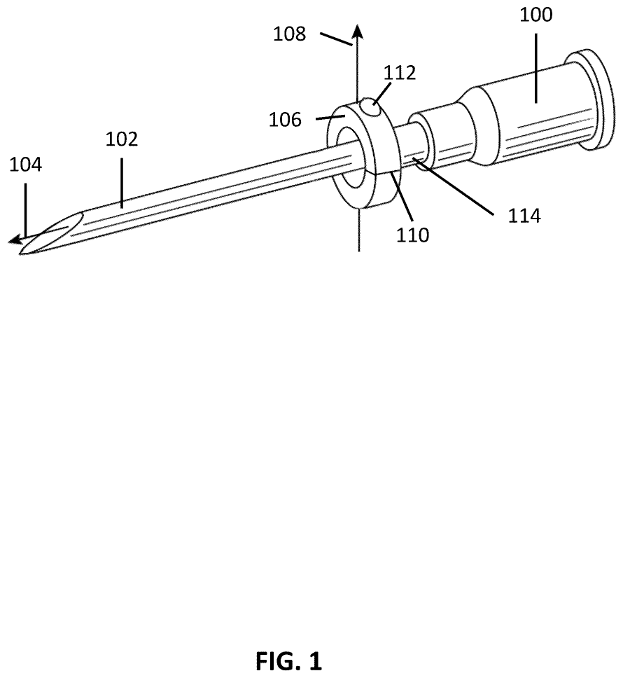

In a non-limiting embodiment illustrated in FIG. 1, the medical device includes a hub 100 positioned proximally relative to a cannula (e.g., needle or catheter shaft) 102 that extends distally along a longitudinal axis 104. A ring-shaped magnetic element 106 can be positioned around the cannula 102, the magnetic element 106 defining a magnetic axis 108 that is orthogonal or substantially orthogonal to the longitudinal axis 104 of the cannula 102. The magnetic element 106 is depicted as a permanent magnet having diametric magnetization, with a magnetic neutral plane 110 orthogonal to the magnetic axis 108 and separating the magnetic element 106 into respective North and South poles. A fiducial 112 can be provided on the exterior of the hub or magnetic element housing to indicate the rotational orientation of the magnetic element 106 and its magnetic axis 108, providing feedback to the user via tactile, colorimetric, visual, or textured means. The magnetic element 106 can be supported by a rotational sleeve 114 that permits free rotation of the magnetic element 106 about the longitudinal axis 104 of the cannula 102. As illustrated, in aspects, the magnetic element 106 may be placed on, attached to, assembled with, and/or assembled onto, the medical device without requiring a pre-oriented, fixed angular orientation of the magnetic axis 108, allowing the magnetic element to be mounted in an arbitrary rotational position relative to the hub or cannula.

The rotational freedom of the magnetic element 106 and its non-coaxial magnetic axis 108—without being required to position the magnetic element in such a way that its magnetic axis is aligned in a pre-determined orientation relative to the hub or needle—provide a measurable asymmetry in the magnetic field that can enable enhanced tracking and calibration accuracy. As the magnetic element 106 rotates, the one or more magnetic field sensors, such as one or more magnetic sensor arrays, can detect predictable variations in field strength and direction, which can be modeled to determine both the spatial position and angular orientation of the needle 102. In embodiments, because the magnetic axis 108 is not aligned with the longitudinal axis 104 of the needle, each rotational position of the magnetic element 106 produces a unique magnetic signature distinguishable by the sensing system. This magnetic element and configuration eliminate the need for fixed angular alignment of the magnetic element during assembly and permits, in cases, automatic orientation recognition by the tracking system upon initialization. In embodiments, deliberate rotation of the magnetic element 106 through known angular intervals may serve as a calibration gesture, allowing the device and/or system to measure field changes attributable to motion of the magnetic element and to subtract constant background components, such as the Earth's magnetic field. This dynamic calibration improves accuracy across varied clinical environments while reducing the need for specialized magnetic shielding or factory alignment procedures, by way of example.

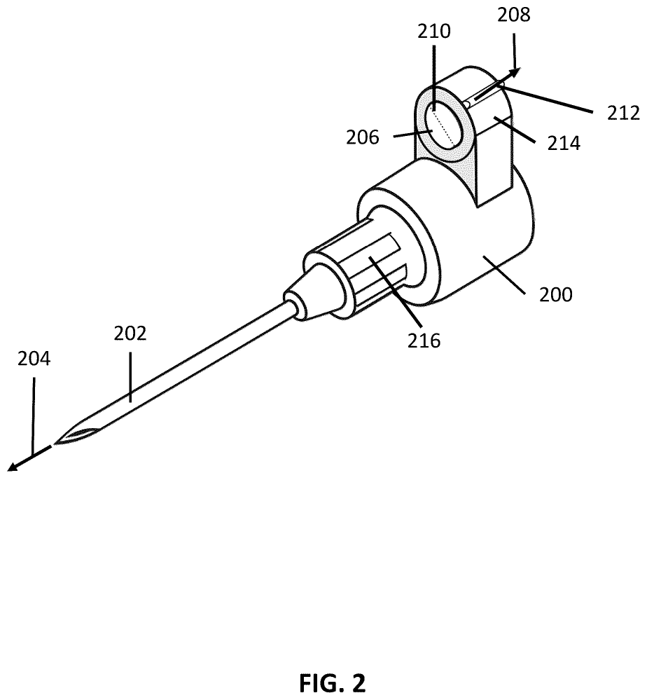

In a non-limiting embodiment illustrated in FIG. 2, the medical device includes a hub 200 positioned proximally relative to a needle 202 that extends distally along a longitudinal axis 204. A cylindrical magnetic element 206 can be attached to, positioned within, located near, located on, or located next to the hub 200, offset from the longitudinal axis 204 of the needle. The magnetic element 206 defines a magnetic axis 208 that is non-coaxial with the longitudinal axis 204 of the needle cannula 202. The magnetic element 206 is depicted as a permanent magnet having diametric magnetization, with a magnetic neutral plane 210 orthogonal to the magnetic axis 208 and separating the magnetic element 206 into respective North and South poles. A physical fiducial 212 can be provided on the exterior of the hub or magnetic housing to indicate the rotational orientation of the magnetic element 206 and/or its magnetic axis 208, providing tactile or visual feedback to the user, for example. The magnetic element 206 can be supported by a rotational sleeve 214 that permits free rotation of the magnetic element 206. As illustrated, the magnetic element 206 may be assembled or combined into or onto the medical device without requiring any fixed angular orientation of the magnetic axis 208, and/or without having to predeterminately position the magnetic axis so that its alignment relative to the hub or needle are predetermined, thereby allowing the magnetic element to be mounted in an arbitrary rotational position relative to the hub or cannula. Rotation of the magnetic element 206 may induce visual changes to a separate indicator 216 not in direct proximity with the magnet via mechanical or other communication of the magnet rotation to a separate indicator that relates the magnetic axis rotational state.

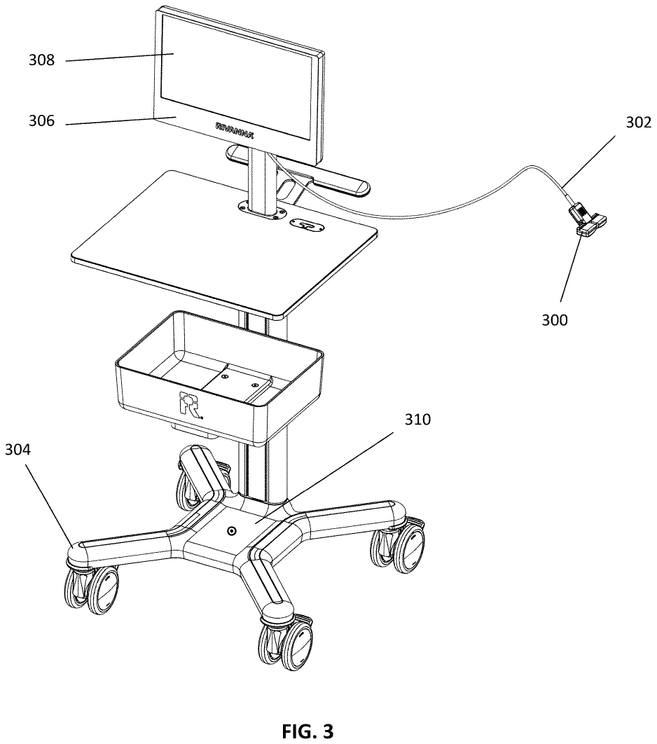

In a non-limiting embodiment illustrated in FIG. 3, a medical ultrasound probe 300 incorporating an integrated magnetic sensor array is operatively connected to an ultrasound imaging system wirelessly or via a probe cable 302. The ultrasound probe can be communicatively coupled to a system processor, such as housed within a computer 306, an electronic device, a remote electronic device, or the cloud, which interfaces with a system display 308 configured to present real-time ultrasound images together with medical instrument position and orientation information derived from magnetic field measurements, as well as other graphical indicators or overlays relevant to the procedure. (The terms “real-time” and “substantially real-time” are used interchangeably herein.) In embodiments, the ultrasound imaging system and associated components may be mounted on a portable medical cart 310 equipped with mobility wheels 304 to facilitate transport and use within various clinical environments.

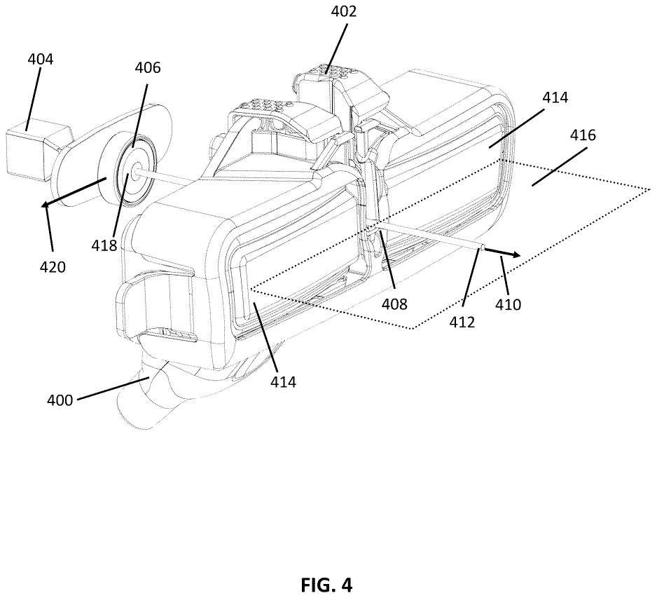

In a non-limiting embodiment illustrated in FIG. 4, an ultrasound probe 400 is configured with an aperture or slot 402 serving as a medical instrument guide for a magnetically trackable medical instrument. The medical instrument can, in aspects, include a hub 404, a magnetic element 406, a cannula (e.g., a needle or a catheter shaft) 408 extending along a longitudinal axis 410, and a distal tip of the cannula 412. The ultrasound probe can include one or more ultrasound transducer arrays 414 that generate a two-dimensional (and/or a three-dimensional) ultrasound image aligned with a defined imaging plane 416. The medical instrument guide aperture or slot 402, optionally incorporated in a sterile attachment to the ultrasound probe 400, can be configured to align the cannula 408 with the imaging plane 416, while still allowing for user-controlled angulation of the medical instrument both within and outside the imaging plane 416. The imaging system can be operatively coupled to the ultrasound probe 400 to perform calculations and/or processes that maintain spatial registration between the ultrasound image and the magnetic sensing volume, thereby enabling accurate, real-time correlation of the needle's and/or needle tip's position and orientation relative to a displayed ultrasound image.

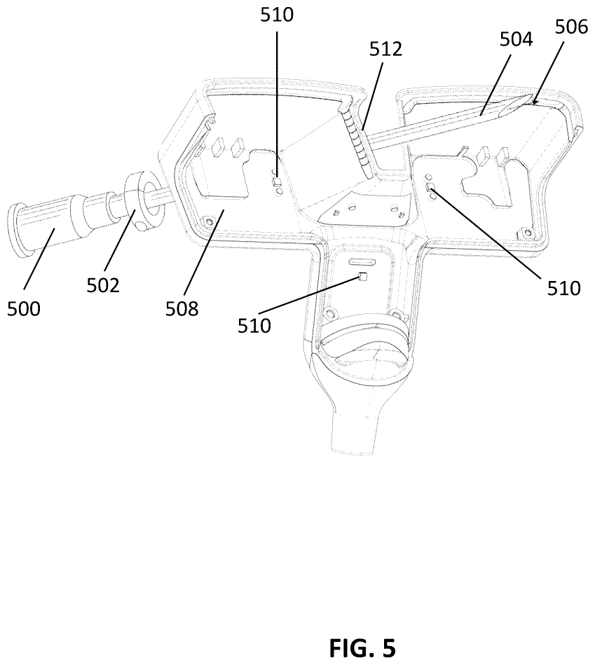

Another non-limiting embodiment is illustrated in FIG. 5. In operation, the three-dimensional magnetic field generated by the magnetic element 502 can be measured by an array of magnetic sensors 510, which produce vector field data corresponding to magnetic flux density at known spatial locations. The array of magnetic sensors 510 may be disposed within the housing of a medical device 508, such as an ultrasound probe, that includes an aperture or guide channel 512 for receiving the needle cannula 504 and directing it along an insertion axis into a patient's body or patient anatomy. In other similar embodiments, the magnetic sensors 510 may be disposed within a medical device that is affixed to, attached to, near, or adjacent to, a patient anatomy to provide tracking of the magnetically trackable medical device relative to the patient anatomy. In embodiments, a control processor receives data from the magnetic sensors 510 (e.g., magnetic sensor array) and computes the spatial position and angular orientation of the magnetic element 502 by analyzing and/or determining magnetic field information that models the magnetic element as a dipole or multipole source defined by its magnetic axis and/or magnetic moment. In embodiments, once the position and rotational state of the magnetic element 502 are established, the processor determines the location of the distal tip of the cannula based, for example, on a known offset distance along the longitudinal axis 506 between the magnetic element 502 and the distal end of the cannula 504. Using this relationship, the processor can derive the medical instrument trajectory, defined as the vector path extending from the hub 500 through the distal cannula tip along the longitudinal axis 506, and continuously update the position, orientation, and/or trajectory of the needle in real time. The computed spatial data may be rendered on a display as an overlay on a live ultrasound or other imaging modality, enabling visualization of the needle tip, cannula, and insertion trajectory relative to anatomical structures or regions. This real-time tracking capability provides spatial feedback to the clinician, improving targeting precision, procedural efficiency, and overall control during needle placement and adjustment.

The known offset distance between the magnetic element 502 and the distal tip of the cannula 504 may be determined during manufacturing, calibration, or initialization of magnetic tracking of the needle. In embodiments, this offset distance is measured mechanically as the linear spacing along the longitudinal axis 506 between a reference point on the hub 500, the physical center of the magnetic element 502, or the magnetic neutral plane and the distal tip. Alternatively, the offset distance may be determined electronically or algorithmically by correlating the magnetic field position data from the magnetic sensors 510 when the distal tip location is placed in a known orientation relative to the magnetic sensors 510. Once established, the offset distance remains constant for that specific medical instrument (e.g., needle) and is stored within the system's calibration data for use in subsequent tracking operations (or during the same medical procedure). Notably, this offset distance is a geometric property of the medical instrument (e.g., needle) and can be independent of the rotational orientation or magnetic axis direction of the magnetic element 502, such that rotation of the magnetic element does not alter (or only insubstantially alters) the spatial relationship between the magnetic element and the distal tip of the needle. As a result, in aspects, accurate needle tip localization and trajectory determination can be achieved regardless of the angular position of the magnetic element 502 about the cannula or its magnetic axis orientation in space.

In embodiments, the position of the distal tip of the needle may be calculated from the measured position of the magnetic element 502 using a known geometric offset between the magnetic element and the distal tip of the needle. The magnetic sensors 510 can provide data from which the processor can determines the three-dimensional coordinates of the magnetic element 502 and the orientation of its magnetic axis. The processor can then apply a geometric model to derive the needle's distal tip position according to a linear relationship of the form pₜᵢₚ=pₘ+dâ, where pₘ represents the position of the magnetic element 502, â is a unit vector corresponding to the longitudinal axis 506 of the cannula 504, and dis the known offset distance between the magnetic element and the distal tip. The calculated offset is a geometric property of the needle and/or the medical device (e.g., by way of example, the needle, hub, magnetic element, and sensors) and remains independent of the rotational state of the magnetic element 502 about the longitudinal axis or its magnetic axis orientation, thereby ensuring consistent needle tip localization during tracking and rotation of the magnetic element.

In embodiments, the cannula of the needle (or catheter shaft) can be sufficiently rigid. In cases, bending of the cannula may reduce the accuracy of tracking. Such a relationship can be maintained by using, for example only, steel or metal cannulas of sufficient flexural modulus to resist bending, the introduction of rigid stylets or guidewires in more flexible materials, such as catheters or thin needles, or the use of needle introducers for preparing a straight path into the patient anatomy for more flexible cannula or instruments.

In embodiments, the magnetic sensors (e.g., magnetic sensor array) utilized by the device and/or system may comprise one or more of magnetometers, Hall-effect sensors, anisotropic magnetoresistive (AMR) sensors, tunneling magnetoresistance (TMR) sensors, or fluxgate sensors, or combinations thereof. Each type of sensor may be selected or arranged according to the desired sensitivity, bandwidth, noise characteristics, or spatial resolution of the magnetic field measurement. In embodiments, a Hall-effect sensor array may be employed to provide compact measurement of the magnetic field along orthogonal axes, suitable for integration directly within the housing of an ultrasound probe or handheld device, by ways of example. In other embodiments, AMR sensors may be used, offering sensitivity and linearity for detecting weak field changes associated with small rotations of the magnetic element. TMR sensors may alternatively be implemented for signal-to-noise performance and, in cases, lower power consumption, particularly in miniaturized sensor assemblies embedded, for example, within the probe body. In embodiments, a fluxgate magnetometer may be used for field measurement, offering thermal stability and low drift, advantageous for calibration or reference field compensation. The device and/or system may employ a hybrid sensor architecture, in which multiple sensor types are co-located or spatially distributed in and/or around the imaging transducer and/or ultrasound probe, for example, to optimize angular resolution and compensate for field distortion effects. The sensors may be arranged in two-dimensional or three-dimensional arrays, enabling the processor to reconstruct the magnetic field vector distribution around the hub and/or needle and to compute the position and orientation of the magnetic element in real time. In embodiments, sensor data may be sampled synchronously with the ultrasound imaging frame rate, ensuring or improving capabilities related to temporal alignment between magnetic tracking data and image acquisition. Through the use of these magnetic sensor technologies, individually or in combination, the system can achieve detection of both static and time-varying magnetic fields associated with the needle, hub, and/or needle tip, facilitating localization and/or gesture-based control under diverse operating conditions.

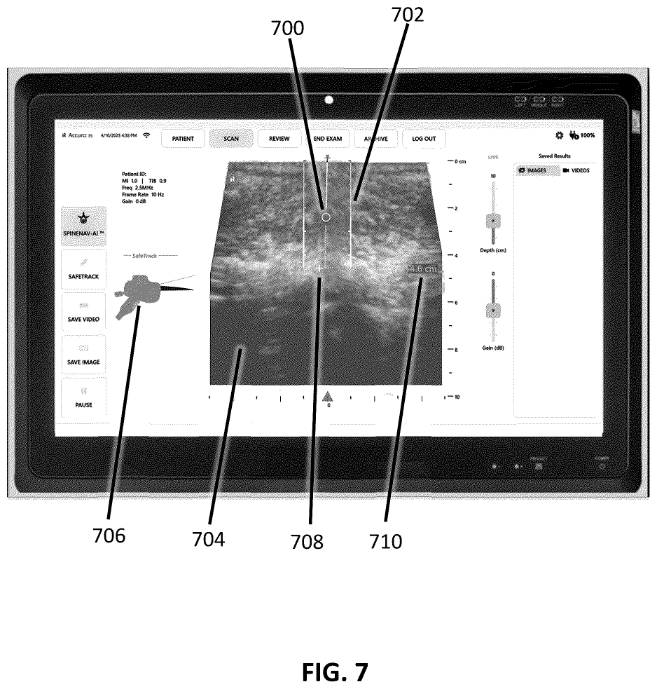

In a non-limiting embodiment illustrated in FIG. 7, the system can display on an electronic display a real-time graphical overlay representing the two-dimensional (2D) projection of the needle's distal tip and associated trajectory guide lanes 702, superimposed upon a live ultrasound image 704. In embodiments, the display may further include a calculated estimate of the distal tip's out-of-plane position 706, presented, in embodiments, as a rendered computer-aided design (CAD) model or symbolic icon that visually conveys the spatial relationship between the distal tip and the ultrasound probe, thereby facilitating navigation and depth perception as a user guides the needle's distal tip to and/or into patient anatomy. In embodiments, the visual icon representing the distal tip's out-of-plane position may be co-located with the distal tip indicator 700 and display either a relative or absolute measure of the out-of-plane distance, for example, by using a circular indicator that increases or decreases in size as the distal tip moves out of or into the imaging plane, respectively. The system may also optionally present one or more anatomical target indicators 708 corresponding to desired needle insertion sites, together with measurement indicators 710 that depict the distance between the distal tip and the designated target anatomical region. In embodiments, the system can generate proximity-based trajectory guidance lanes 702 incorporating a zone-based countdown indicator that communicates the relative position of the distal tip to the anatomical target region 708, such as through visual cues interpreted as “far,” “closer,” or “close,” for example only. These indicators can assist the user in maintaining spatial awareness and avoiding sensitive structures. The zone-based cues may be presented as on-screen graphical elements 708 and/or as synchronized light-emitting diodes (LEDs) or equivalent indicators integrated, for example, within the probe housing, enabling the user to monitor needle and/or needle tip proximity without diverting visual attention from the procedural field of view. In embodiments, the system may be configured to automatically detect anatomical target regions 708 within the ultrasound image and provide a depth estimate 710 to each identified target region, using, for example, template matching, artificial intelligence, or machine learning techniques. The depth, and anatomic target regions and/or positions, may then serve as reference parameters for countdown and/or proximity indicators, providing visual and/or auditory guidance throughout the needle insertion process. Automated detection and distance estimation can serve to improve standardization, reduce variability associated with operator-defined targets, and enhance the overall accuracy and efficiency of the image-guided tracking workflow.

According to embodiments of the present invention, the magnetic axis of the magnetic element is not oriented coaxially with the longitudinal axis of the needle cannula, thereby producing an asymmetric magnetic field that varies predictably as the element is rotated. Such non-coaxial configurations can enable determination of both angular and positional information from the detected magnetic field. In embodiments, the magnetic element may comprise an obliquely magnetized cylindrical magnet, wherein the magnetization vector is skewed relative to the geometric centerline of the cylinder. This orientation generates a field having both axial and lateral components that vary measurably with rotation. In another embodiment, the magnetic element may be a diametrically magnetized cylinder or ring, in which opposite poles are positioned on the lateral surfaces of the magnet to produce a transverse or substantially transverse field relative to the cannula longitudinal axis, providing an orthogonal instance within the class of non-coaxial magnetic geometries. In further embodiments, the magnetic element may be a radially magnetized ring magnet, magnetized either inward or outward to generate a circumferentially distributed magnetic field that interacts with one or more sensors and/or one or more magnetic sensor arrays disposed around, in, near, on, or below the hub. In yet another configuration, a segmented and/or composite multi-pole magnet may be employed, in which multiple magnet segments of alternating polarity are arranged along a circle or arc, the composite field of which exhibits a dominant non-coaxial component defined by the vector sum of the individual magnetic moments. In these embodiments, a laterally offset magnet placement may be implemented, wherein a cylindrical or disk magnet is mounted eccentrically within the hub (or outside the hub) such that its magnetic center is displaced from the cannula axis. In other embodiments, the magnetic element may comprise an electromagnet or coil energized to generate a magnetic field oriented obliquely or transversely relative to the cannula axis, with the coil optionally configured for controlled or rotatable operation to dynamically alter field orientation. A hybrid permanent-electromagnetic design may also be used, combining a baseline permanent magnetic field with a supplementary electromagnetic adjustment field to modify magnitude or direction of the resultant non-coaxial field. Each of these embodiments produces a rotationally asymmetric magnetic field, wherein vector direction changes with rotation of the magnetic element, enabling magnetic sensors, such as tri-axial magnetic sensors, to measure angular displacement, rotational state, and positional information of the needle in real time.

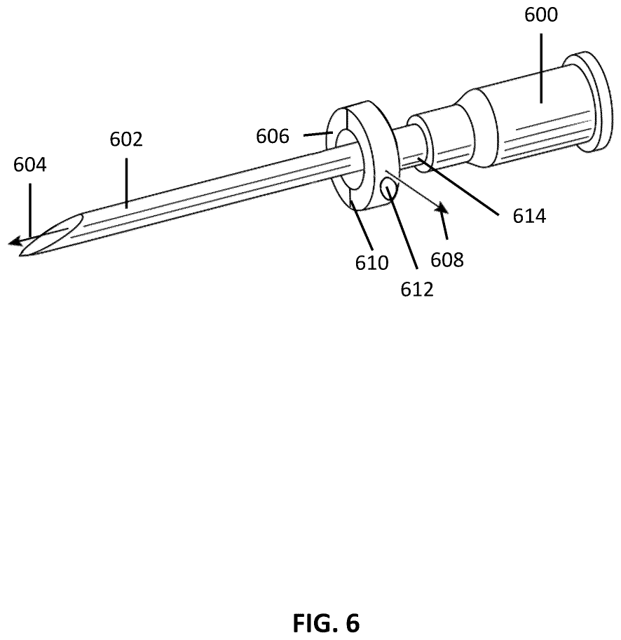

In a non-limiting embodiment illustrated in FIG. 6, the medical instrument includes a hub 600 positioned proximally relative to a cannula 602 that extends distally along a longitudinal axis 604. A ring-shaped magnetic element 606 defines a magnetic axis 608 that is orthogonal or substantially orthogonal to the longitudinal axis 604 of the cannula 602, generating a magnetic field that is transverse or substantially transverse to the longitudinal axis 604 of the cannula 602. The magnetic element 606 is depicted as a permanent magnet having diametric magnetization, with a magnetic neutral plane 610 orthogonal to the magnetic axis 608 and separating the magnetic element 606 into respective North and South poles. A fiducial 612 can be provided on the exterior of the hub or magnetic housing to indicate the rotational orientation of the magnetic element 606 and its magnetic axis 608, providing feedback to the user via tactile, colorimetric, visual, or textured means. Relative to the non-limiting embodiment depicted in FIG. 1, the magnetic element 606 in FIG. 6 has been rotated by approximately 90 degrees, resulting in the magnetic axis 608 occupying a new rotational state that may be detected by an array of magnetic sensors, for example, to perform functions described in the various embodiments described herein.

In alternative embodiments, the rotational functionality of the magnetic element may be achieved through a variety of mechanical, electromechanical, or material configurations other than the rotational sleeve described herein. In embodiments, a laterally offset magnet assembly is provided, wherein the magnetic element is mounted to one side of the cannula within the hub. This off-axis configuration produces a non-coaxial magnetic component and, in cases, can simplify manufacturing while preserving rotational sensitivity. In embodiments, a dual oblique magnet pair may be employed, in which two magnets are embedded within the hub at different oblique orientations to generate a composite asymmetric field. The resultant vector sum of the individual magnetic fields can, in cases, enhance detection sensitivity, improve redundancy, and permit angular determination by an external sensor and/or sensor array. In yet another embodiment, an adjustable magnet module may be utilized, comprising a rotatable cartridge or insert containing the magnetic element. The module may incorporate fiducial markings and a locking collar to permit controlled user rotation, positional indexing, or temporary fixation during use. In embodiments, the magnetic element can be supported within an elastomeric bearing, wherein a compliant material seat allows limited rotational motion while accommodating fine or more fine calibration movement under user torque. In an electromagnet variant, the permanent magnet is replaced with a coil wound around a ferromagnetic core oriented at an oblique angle relative to the cannula axis. When energized, the coil produces a dynamic magnetic field that can alternate direction or polarity to facilitate calibration, self-verification, or communication signaling with external sensors. In another embodiment, a hybrid module is provided in which a permanent magnet is co-located with an electromagnetic coil within the hub, the coil being configured to bias or perturb the static magnetic field of the permanent magnet in a controlled manner for signal encoding or field verification purposes. In embodiments, the magnetic assembly may be fabricated as an encapsulated module, such as a sterile module, wherein the magnetic element and rotational components are sealed within a polymer housing integrated with or placed near, in, or on, a disposable hub or connector assembly for sterility and to maintain operational functionality. In a further embodiment, a self-sensing magnet and/or self-sensing magnet assembly may be implemented, incorporating an internal magnetic sensor within the hub to directly measure the orientation of the magnetic element's own field, thereby providing closed-loop feedback for alignment verification and/or calibration without reliance solely on external field measurements. Each of these embodiments provides alternative means for enabling rotation or dynamic orientation of the magnetic element while maintaining the asymmetric, non-coaxial magnetic characteristics for field detection and positional tracking.

In various embodiments, the processor and/or tracking system associated with the magnetic sensing array can be configured to interpret changes in the rotational state of the magnetic element as intentional user inputs. Rotational motion of the magnetic element produces a corresponding variation in the measured magnetic field vector, which can be analyzed by the processor to determine both the direction and magnitude of angular displacement. When a defined rotational event, such as a rotation exceeding a specified angular threshold or passing through a predefined detent, is detected, the processor may register the motion as a command input. In this manner, user rotation of the magnetic element may be employed to initiate or terminate specific system operations, including activation of needle tracking, marking the start or end of procedural phases, toggling display overlays, advancing between interface states, or initiating calibration routines. The processor and/or tracking system may further associate a discrete rotational increment(s) with a function or state change, providing a sterile-field-compatible interface that eliminates or reduces the need for contact with external controls. In embodiments, the rotational input is accompanied by a corresponding visual indicator on the display that depicts the magnetic axis orientation or the selected operational state, so that the user receives feedback confirming the executed command. This integration of magnetic sensing and/or gesture-based input provides a means of procedural interaction, enabling, in embodiments, efficient, hands-free control of device and/or system functions during real-time image-guided interventions.

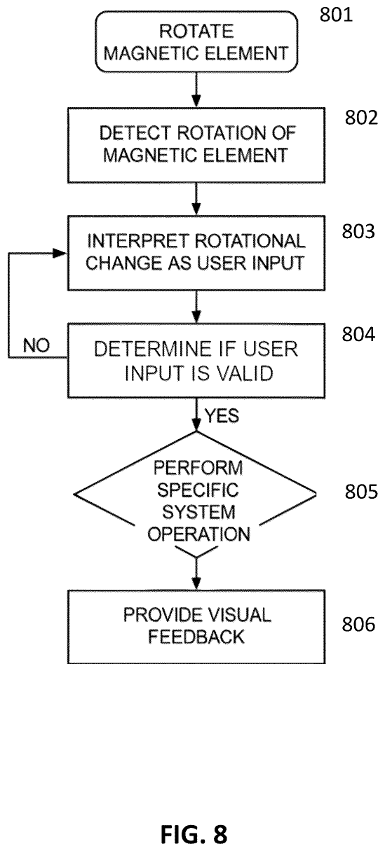

An exemplary flowchart of a non-limiting embodiment of a magnetic-gesture-based user-input process is illustrated in FIG. 8. In certain embodiments, a rotational mechanism of the magnetic element can be configured to provide user feedback corresponding to angular positions or motion thresholds. For example, the hub or magnetic housing (e.g., containing part or all of the magnetic element) may incorporate mechanical detents, haptic clicks, or torque-resistive features that deliver tactile or audible confirmation as the magnetic element is rotated 801 through angular increments (e.g., predefined angular increments)—such as 45°, 90°, or 180°. These feedback features can enable the user to perform intentional rotational gestures that are detected 802 by the magnetic sensors and interpreted by the system processor as user-interface inputs 803. When the system identifies a confirmed and valid user input by comparing to an action threshold or input pattern 804, by way of example, the system will initiate or otherwise perform the system operation desired by the user according to the user input 805. In some embodiments, the rotational resistance may be modulated in a controlled manner to indicate completion of calibration routines or to minimize inadvertent movement during normal handling. The feedback mechanism may be implemented using spring-loaded pawls, elastomeric ridges, magnetic couplings, or indexed collars, designed to preserve sterility while ensuring safe and repeatable motion. In embodiments, the tactile or audible responses are synchronized with on-screen visual indicators 806 corresponding to the magnetic axis orientation, providing multi-modal feedback that reinforces user awareness of the magnetic element's angular state. Collectively, these feedback mechanisms can improve ergonomic usability and procedural confidence, enabling the user to perform calibrated rotational inputs that correspond to specific system functions/operations 805—such as activating magnetic tracking, marking procedural events, or adjusting display modes 807—without the need to contact non-sterile external controls.

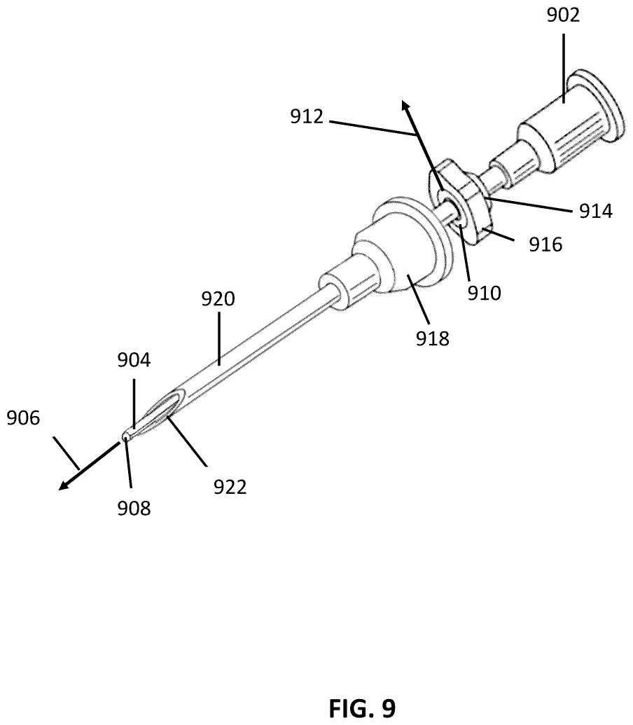

In a non-limiting embodiment illustrated in FIG. 9, the medical instrument can be a catheter comprising a hub 902, a cannula 904 defining a longitudinal axis 906, and a distal tip 908. The catheter can further include a magnetic element 910 comprising a magnetic axis 912 supported, in this example, by a rotational sleeve 914, which is configured to permit free rotation of the magnetic element 910 about the longitudinal axis 906 of the cannula 904. The rotational sleeve 914 can include fiducials 916 that indicate the rotational state of the magnetic element 910 to the user and provide surfaces that allow manual manipulation of the magnetic element's rotational position. In embodiments, the catheter may be used in conjunction with an introducer needle comprising a separate hub 918, a cannula 920, and a distal tip 922, optionally configured with a cutting edge to facilitate percutaneous entry of the medical instrument into the body.

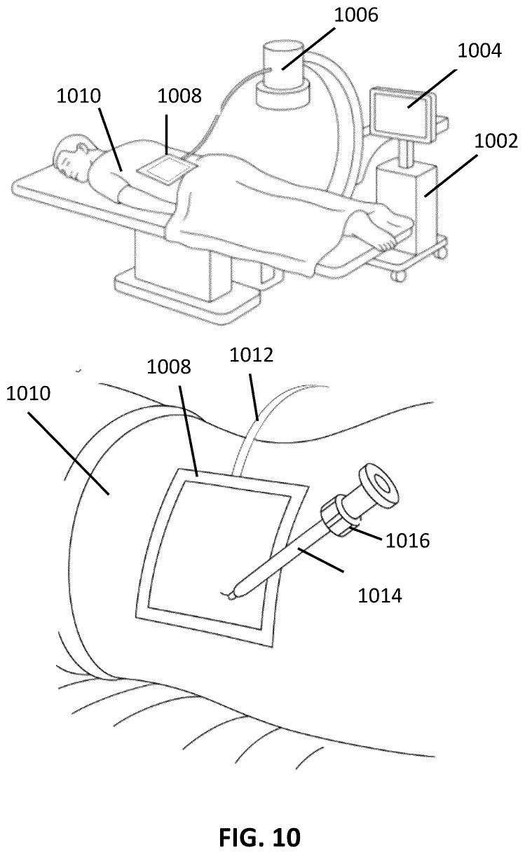

In a non-limiting embodiment illustrated in FIG. 10, a medical imaging system 1002 is provided that is configured to display positional, tracking, guidance, and/or imaging information, associated with a magnetically tracked medical instrument. The system 1002 includes a display 1004 and one or more imaging sensors 1006 operatively coupled to a component 1008 that comprises an array of magnetic sensors configured to detect a magnetic field generated by a magnetic element of the tracked medical instrument.

In embodiments, the component 1008 is positioned in direct contact with a patient 1010, while in other embodiments, the component 1008 is positioned in proximity to the patient—for example, adjacent to or near the anatomical region where needle insertion or other interventional activity is occurring or is planned to occur—such that magnetic field detection can be achieved without direct physical contact.

The component 1008 can be operatively connected to the imaging system 1002 via a data cable 1012, although in alternative embodiments, the connection may be established wirelessly. The magnetically tracked medical instrument 1014 (e.g., a needle) can include a rotatable magnetic element 1016. The magnetic element 1016 generates a magnetic field that is detected by the array of magnetic sensors within or associated with the component 1008, or wherein the component comprises one or more magnets or arrays of magnetic sensors.

The sensed magnetic field data can be processed by the medical imaging system 1002 to determine the spatial position and orientation of the medical instrument 1014 relative to the patient 1010 for tracking purposes, by way of example. In embodiments, the system may further be configured to display a real-time visual representation of the tracked instrument on the display 1004, optionally overlaid with anatomical or imaging data to assist with tracking, navigation, and/or procedural accuracy.

In embodiments, the rotational movement of the magnetic element may serve a dual function as both a user-interface input and a magnetic calibration mechanism. In aspects, when the magnetic element is intentionally rotated through a predefined angle, the resulting time-varying magnetic field data can be analyzed by the processor to identify and isolate static or slowly varying background magnetic components, such as the Earth's magnetic field or bias fields produced by nearby ferromagnetic materials. By comparing the measured field variations during rotation with an idealized dipole model of the magnetic element, the system can compute and remove offset values corresponding to these static influences, effectively zeroing (or substantially zeroing) the sensing baseline. In embodiments, the processor can perform this calibration automatically upon detection of a continuous or predefined rotational motion, whereas in other embodiments, the user may intentionally initiate the calibration sequence by rotating the magnetic element through a specific angular pattern or sequence of detent positions. This calibration process may occur prior to or during a procedure and does not require any external reference field or manual sensor alignment, in aspects. In some cases, the calibration relies solely on the relative change in the magnetic field rather than its absolute orientation, such that the process is independent of the magnet's mounting orientation, position, or rotational starting state. Once calibration is complete, the processor can update internal field compensation parameters to enhance positional accuracy of the needle tip and trajectory tracking, thereby improving system robustness across different clinical environments and magnetic conditions.

In aspects, following calibration, the processor can apply the updated compensation parameters to subsequent magnetic field measurements to maintain real-time accuracy of needle localization and visualization. The corrected field data can be, in aspects, continuously processed to determine the three-dimensional position and angular orientation of the magnetic element, from which the needle tip position, insertion trajectory, and bevel orientation are derived. These computed spatial parameters can then be mapped to the imaging coordinate frame of the medical imaging modality, such as an ultrasound probe or system, improving a process of registration between the magnetic tracking data and the live anatomical image. The system can dynamically update this registration as the needle moves or rotates, maintaining accurate alignment even during patient motion or probe repositioning. On the display, the processor can render a graphical overlay depicting one or more of the needle distal tip, needle shaft, and projected path relative to the ultrasound image or other imaging modality. In embodiments, the overlay may include a symbolic indicator of the magnetic axis orientation corresponding to the current rotational state of the magnetic element, thereby providing the user with real-time visual confirmation of both positional and angular alignment. The integration of continuously calibrated magnetic tracking with real-time imaging can enhance the spatial fidelity of the system, allowing the user to visualize the instrument's path toward the target anatomical region with improved depth perception, reduced alignment error, and greater procedural confidence.

In embodiments, the system processor may determine the three-dimensional position and orientation of the magnetic element by matching the magnetic field measurements obtained from one or more magnetic sensors (or one or more sensor arrays) to a theoretical or empirically derived field model. In embodiments, a brute-force model-matching algorithm may be employed in which the system compares the measured magnetic field vectors to a precomputed dataset representing possible spatial positions and angular orientations of the magnetic element. The processor can identify the model state that minimizes the error metric, such as, for example, the least-squares difference between measured and predicted field values, thereby determining the magnetic element's position and orientation. In another embodiment, the processor may use an iterative optimization algorithm, such as gradient descent, Gauss-Newton, or Levenberg-Marquardt, to converge on the position and orientation parameters that fit (or best or optimally fit) the observed field data relative to the model. In some implementations, a sparse search or coarse-to-fine hierarchical approach may be used to accelerate convergence by first estimating a broad spatial region of interest, followed by localized refinement to submillimeter precision. The underlying magnetic field model used for comparison may be derived through analytical dipole or multipole equations, finite-element simulation, or empirical calibration measurements conducted with the magnetic element at known positions relative to the sensor array. In embodiments, the system may employ a hybrid modeling approach, combining simulation-based predictions with empirically corrected field maps to account for sensor tolerances, housing geometry, or magnetic interference from surrounding materials. The processor may also implement machine-learning or regression-based algorithms trained on empirical datasets to predict position and orientation directly from the sensor vector data, bypassing explicit model equations. In some variants, Kalman filters, particle filters, or extended state observers may be employed to fuse time-sequential sensor measurements, thereby improving accuracy and stability during motion. Each of these computational techniques can enable the system to robustly infer the six degrees of freedom (6DOF)—three translational and three rotational—of the magnetic element in real time, even in the presence of environmental noise or partial sensor occlusion. By flexibly combining analytical, empirical, and data-driven modeling approaches, the system can achieve high-fidelity magnetic localization and orientation tracking across a broad range of clinical and operational conditions.

In embodiments, the system may be configured to record, store, and export data corresponding to the magnetic tracking and imaging operations performed during a procedure. The processor may continuously log the computed three-dimensional position and orientation of the magnetic element, the derived instrument tip location and trajectory, and the rotational state history of the magnetic axis throughout instrument insertion, repositioning, and withdrawal. These data may be time-synchronized with the associated ultrasound image frames and system events, such as user-initiated commands or calibration sequences, to create a procedural record. In embodiments, the recorded data are stored locally within the imaging system (or related device(s)) or transmitted via a wired or wireless connection to an external computing device, hospital network, remote processor, or cloud-based storage system. The stored trajectory and orientation data may subsequently be replayed, analyzed, or visualized to assess procedural accuracy, verify targeting performance, or support training and quality assurance activities. In addition, the system may export standardized data files or reports containing spatial coordinates, calibration parameters, and timestamps for integration with electronic health records or research databases. By preserving a history of magnetic tracking and imaging information, the system can facilitate post-procedure verification, reproducibility, and auditability, while also enabling advanced analytics and system optimization across multiple clinical applications.

In embodiments, the system may utilize the recorded magnetic and imaging data to perform adaptive calibration refinement and predictive guidance through machine learning or statistical modeling techniques. The stored positional, orientation, and calibration data from multiple procedures may be analyzed by a training algorithm to identify systematic offsets, environmental distortions, or user-specific handling patterns that affect tracking accuracy. Based on these analyses, the processor may update internal calibration models or generate correction coefficients that enhance the fidelity of position and orientation estimation in subsequent uses. In embodiments, a predictive guidance subsystem may employ real-time sensor input together with previously learned models to estimate the anticipated trajectory of the tip relative to anatomical target regions, compensating dynamically for motion, tissue deformation, or transient tracking anomalies. The system may further present predictive overlays or trajectory suggestions on the display to assist the user in maintaining alignment with the intended path of insertion. In embodiments, the adaptive algorithms may execute locally within the imaging and/or system processor, while in others, the data are transmitted to a remote server, remote computing device, or cloud-based learning environment that aggregates procedural information from multiple systems to refine performance collectively across an installed base. By leveraging machine learning to continuously improve calibration accuracy and predictive control, the system enables self-optimizing needle tracking performance that becomes increasingly precise and robust with continued clinical use.

In embodiments, the adaptive tracking and predictive guidance capabilities of the system may be configured to provide context-aware safety features that assist in preventing unintended instrument placement or tissue injury. The processor may continuously evaluate the computed or predicted instrument trajectory relative to anatomical structures identified within the ultrasound or other imaging modality. When the trajectory, tip position, or projected path deviates from a designated target anatomical region or approaches a critical anatomical boundary, such as a blood vessel, nerve, or organ capsule, the system may generate an automated alert to notify the user. Such alerts may include visual indicators on the display, audible tones, or haptic feedback delivered through the probe or instrument housing, for example. In embodiments, the system may dynamically adjust the displayed overlay to highlight the deviation or to visually depict a corrective path based on the predicted trajectory. The alert thresholds and target boundaries may be defined automatically through image segmentation algorithms or manually by the user prior to the procedure. In embodiments, the system may utilize stored anatomical models, machine learning models, or learned procedural data to refine its contextual awareness, allowing it to distinguish between intentional and unintentional trajectory changes. Through these integrated safety functions, the system can enhance procedural confidence, reduce the risk of off-target insertions, and provide real-time intelligent assistance during image-guided interventions.

In embodiments, the system may be operatively integrated with a robotic manipulator, navigation platform, or mechanical positioning device configured to assist or automate needle placement based on the real-time tracking, imaging, and predictive guidance data. The processor may transmit continuous updates of the instrument position, orientation, and trajectory vector to a robotic controller, which in turn adjusts the position or alignment of the needle, ultrasound probe, or both, to maintain optimal targeting relative to the anatomical region of interest. In embodiments, the robotic system may operate in a semi-autonomous mode, wherein the user retains manual control of insertion while the robot provides fine motion compensation or positional stabilization to correct for patient movement, breathing, or tissue deformation. In embodiments, the robotic platform may perform fully automated insertions along a calculated trajectory derived from the magnetic and imaging data, using closed-loop feedback from the magnetic sensors to continuously verify alignment and tip position. The communication between the tracking system and the robotic controller may occur via wired or wireless data links, and may incorporate redundancy or safety interlocks to prevent unintended motion. Integration of the magnetic tracking and predictive control functions with robotic actuation enables precise, repeatable, and operator-independent needle guidance, expanding the applicability of the system to advanced image-guided interventions, automated sampling, and targeted therapeutic delivery.

In embodiments, the system may be configured to perform simultaneous tracking and coordination of multiple magnetic elements and/or needles during a single procedure. Two or more needle apparatus, each including an accompanying, respective magnetic element having a distinct magnetic strength, orientation, and/or temporal modulation pattern, may be introduced into the procedural field. (It is envisioned that this could also be performed wherein the accompanying, respective magnetic elements have indistinct magnetic strengths, orientations, and/or temporal modulation patterns.) The array of magnetic sensors can be configured to detect and resolve the superimposed magnetic fields produced by the individual magnetic elements, allowing the processor to independently compute the position, orientation, and trajectory of each needle in real time. In embodiments, the system may employ frequency, phase, or amplitude modulation of the magnetic field—either through rotation of permanent magnets or by alternating current excitation of electromagnets—to distinguish among multiple instruments. In aspects, the processor may render corresponding visual overlays on the display, assigning unique colors, labels, or trajectory indicators to each tracked needle. In embodiments, positional relationships between the tracked needles may be analyzed to support cooperative targeting, such as triangulating a specific anatomical region or maintaining parallel or converging insertion angles. The multi-needle configuration may also enable dual-needle or multi-ultrasound probe workflows, such as simultaneous administration of therapeutic agents, sampling from multiple tissue sites, or combined ablation and monitoring procedures. By enabling concurrent, independent tracking of multiple magnetic elements (and/or needles), the system can provide a scalable foundation for complex, multi-instrument image-guided interventions with enhanced precision and workflow efficiency.

The current invention includes several Aspects:
Aspect 1: A medical device comprising:
a base or hub connected directly or indirectly to a needle or a catheter shaft;
the needle or the catheter shaft comprising a distal tip, wherein the needle or the catheter shaft extends away from the base or the hub and towards the distal tip along a longitudinal axis; and
a magnetic element configured for rotational movement, such that as the magnetic element rotates, the magnetic axis of the magnetic element moves relative to the longitudinal axis of the needle or catheter shaft.
Aspect 2: The medical device of Aspect 1, wherein the magnetic axis of the magnetic element is arbitrarily or randomly oriented relative to the longitudinal axis of the needle or the catheter shaft.
Aspect 3: The medical device of Aspect 1, wherein the rotatable magnetic element is positioned in, on, or near, the hub, the base, the needle, or the catheter shaft, such that the magnetic axis of the rotatable magnetic element is variable, undetermined, feedback-based, or determined in real time or substantially real time.
Aspect 4: The medical device of Aspect 1, wherein the magnetic axis of the magnetic element is transverse, orthogonal, or diagonal, to the longitudinal axis of the needle or the catheter shaft.
Aspect 5: The medical device of Aspect 1, wherein the magnetic axis of the magnetic element is directed across or to otherwise intersect with the longitudinal axis of the needle or the catheter shaft.
Aspect 6: The medical device of Aspect 1 wherein the magnetic element comprises a ring-shaped body having an opening through which at least a portion of the needle or the catheter shaft extends.
Aspect 7: The medical device of Aspect 1, wherein the magnetic element is a cylindrical magnet comprising an opening, wherein the cylindrical magnet is positioned in, on, or near, the needle or the catheter shaft such that the longitudinal axis of the needle or the catheter shaft does not extend through the body of the magnet.
Aspect 8: The medical device of Aspect 1, wherein the magnetic element is disposed about or around the needle or the catheter shaft, such that the magnetic element is rotatable about or around the longitudinal axis of the needle or the catheter shaft.
Aspect 9: The medical device of Aspect 1, wherein the magnetic element is magnetized in a diametric direction such that its magnetic axis is substantially perpendicular or substantially diagonal to the longitudinal axis of the needle or the catheter shaft.
Aspect 10: The medical device of Aspect 1, wherein the magnetic element is retained within a recess, cavity, or sleeve, of the hub or the base, such that the magnetic element is configured for rotational movement relative to the needle or the catheter shaft.
Aspect 11: The medical device of Aspect 1, wherein the magnetic axis of the magnetic element is not aligned in a predetermined orientation relative to the hub, the base, the needle, or the catheter shaft, during assembly or manufacturing of part or all of the medical instrument.
Aspect 12: The medical device of Aspect 1, wherein the magnetic element includes at least one visual, haptic, auditory, or tactile fiducial, indicating an orientation of the magnetic axis of the magnetic element to enable a user of the medical instrument to identify or adjust an angular position of the magnetic element relative to the medical instrument, the needle, or the catheter shaft.
Aspect 13: The medical device of Aspect 12, wherein the at least one visual, haptic, auditory, or tactile fiducial, comprises a raised ridge, a groove, an indentation, a protrusion, a color marking, or a textured surface, which is configured to correlate with a magnetic-axis orientation of the magnetic axis of the magnetic element.
Aspect 14: The medical device of Aspect 12, wherein the at least one visual, haptic, auditory, or tactile fiducial, provides a tactile cue configured to be perceptible by touch, the tactile cue configured to indicate a magnetic-axis orientation of the magnetic axis of the magnetic element during manual or automatic manipulation of the medical instrument, the magnetic element, the needle, or the catheter shaft.
Aspect 15: The medical device of Aspect 12, wherein the at least one visual, haptic, auditory, or tactile fiducial, provides a visual cue configured to be visible through a clear, transparent, or translucent portion of the hub or the base.
Aspect 16: The medical device of Aspect 12, wherein the at least one visual, haptic, auditory, or tactile fiducial, is mechanically keyed to a magnetic-axis orientation of the magnetic axis of the magnetic element to maintain a fixed angular relationship therewith.
Aspect 17: The medical device of Aspect 1, further comprising a rotational sleeve, bearing, or bushing, disposed between the magnetic element and the hub, the base, the needle, or the catheter shaft, to facilitate the rotational movement of the magnetic element.
Aspect 18: The medical device Aspect 1, further comprising a sensor or wherein the medical instrument is in wired or wireless communication with a sensor, wherein the magnetic element generates a transverse magnetic field detectable by the sensor, and wherein the sensor is configured to determine an orientation or a position of the medical instrument, the needle, the catheter shaft, the distal tip of the needle, or the distal tip of the catheter shaft.
Aspect 19: The medical device of Aspect 1, wherein the magnetic element comprises a permanent magnet.
Aspect 20: The medical device of Aspect 1, wherein the magnetic element comprises an electromagnet configured to generate a magnetic field in response to an applied current.
Aspect 21: The medical device of Aspect 1, further comprising a sensor or wherein the medical instrument is in wired or wireless communication with a sensor, the sensor configured to detect a rotational position of the magnetic axis of the magnetic element relative to the longitudinal axis of the needle or the catheter shaft.
Aspect 22: The medical device of Aspect 21, wherein the sensor is further configured to continuously or periodically track the rotational position of the magnetic axis of the magnetic element over time.
Aspect 23: The medical device of Aspect 1, wherein the magnetic element is completely or partially rotatable before or during a medical procedure, wherein the needle or the catheter shaft are inserted into a patient or a patient anatomy during the medical procedure.
Aspect 24: A system for magnetic-based medical instrument tracking or guiding, comprising:
a base or a hub and a needle or catheter shaft extending distally from the base or the hub along a longitudinal axis, the needle or the catheter shaft comprising a distal needle tip or catheter shaft tip;
a magnetic element having a magnetic axis that varies rotationally relative to the longitudinal axis of the needle or the catheter shaft, wherein the magnetic element is associated with or directly or indirectly connected to the base, the hub, the needle, or the catheter shaft; and
a medical imaging device comprising:
one or more processors;
one or more imaging sensors configured to acquire image and/or image data of an anatomical region; and
a plurality of electronic magnetic sensors (a) disposed on the medical imaging device, (b) disposed within the medical imaging device, or (c) in operative communication with the one or more processors;
wherein the one or more processors are configured to measure a magnetic field generated by the magnetic element in three dimensions to determine a location, an orientation, or both, of the medical instrument, and display a composite overlay of the determined location, orientation, or both, of the medical instrument along with one or more images of the anatomical region based on the acquired image and/or image data of the anatomical region.
Aspect 25: The system of Aspect 24, wherein the medical imaging device is configured for one or more of the following imaging modalities: ultrasound, fluoroscopy, x-ray, computed tomography, MRI, or optical.
Aspect 26: The system of Aspect 24, wherein the composite overlay further includes at least one indicator representing the position, the orientation, a trajectory, or combinations thereof, of the needle, the catheter shaft, the needle tip, or the catheter shaft tip
Aspect 27: The system of Aspect 24, wherein the one or more processors, and a computer-readable medium having executable instructions stored thereon, such that, when the executable instructions are executed by the processor, the processor is operative to receive information from the plurality of electronic magnetic sensors and, in response to the signals: output instructions; output information related to a position of the needle, the catheter shaft, the needle tip, or the catheter shaft tip; output information related to an orientation of the needle, the catheter shaft, the needle tip, or the catheter shaft tip, or combinations thereof, to a user of the system.
Aspect 28: The system of Aspect 24, wherein the one or more processors, and a computer-readable medium having executable instructions stored thereon, such that, when the executable instructions are executed by the processor, the processor is further operative to generate and display, in real time or substantially real time, the composite overlay of the determined location, orientation, or both, of the medical instrument along with the one or more images of the anatomical region based on the acquired image and/or image data of the anatomical region.
Aspect 29: The system of Aspect 24, wherein the composite overlay further includes one or more of, a depiction of the needle, a depiction of the catheter shaft, a depiction of the needle tip, a depiction of the catheter shaft tip, a predicted or suggested needle or catheter shaft trajectory, or a predicted or suggested needle tip or catheter shaft tip trajectory, which are optionally updated on the composite overlay in real time or substantially real time.
Aspect 30: The system of Aspect 24, wherein the plurality of electronic magnetic sensors surround or substantially surround a portion of the longitudinal axis of the needle or the catheter shaft, and wherein the plurality of electronic magnetic sensors and the portion of the longitudinal axis of the needle or the catheter shaft are optionally located within the medical imaging device or optionally directly or indirectly connected to the medical imaging device.
Aspect 31: The system of Aspect 24, wherein the medical imaging device is an ultrasound imaging device, wherein the ultrasound imaging device includes a probe body, and wherein the probe body includes two ultrasound arrays housed within the probe body, and wherein the longitudinal axis of the needle or the catheter shaft is positioned between the two ultrasound arrays.
Aspect 32: The system of Aspect 24, wherein the one or more processors, and a computer-readable medium having executable instructions stored thereon, such that, when the executable instructions are executed by the processor, the processor is further operative to perform a magnetic calibration routine configured to measure and optionally cancel one or more of, contributions of static or substantially static magnetic fields, magnetic interference, one or more stray magnetic field, magnetic noise, or external magnetic disturbance, by detecting changes in the magnetic field during a user-induced rotation of the magnetic element.
Aspect 33: The system of Aspect 24, wherein the magnetic element includes at least one visual, auditory, or tactile fiducial, corresponding to or indicating a rotational orientation of the magnetic axis.
Aspect 34: The system of Aspect 24, wherein the composite overlay includes graphical representation of the position, the orientation, or both, of the needle, the catheter shaft, the needle tip, or the catheter shaft tip.
Aspect 35: The system of Aspect 24, wherein rotation of the magnetic element relative to a sensing geometry of the plurality of electronic magnetic sensors is detected by and interpreted by the one or more processors as a user-interface command input.
Aspect 36: The system of Aspect 24, wherein a user-manipulated rotation of the magnetic element through a predefined angular increment is configured to cause the one or more processors to execute a predefined function.
Aspect 37: The system of Aspect 24, wherein the predefined function comprises one or more of:
(a) initiating needle or catheter shaft tracking mode;
(b) marking a start and/or an end of a procedure phase or event during which the medical instrument and/or system are used;
(c) recording a time interval between the two procedural phases or events; or
(d) toggling a calibration of a displayed composite overlay state.
Aspect 38: The system of Aspect 24, wherein the one or more processors, and a computer-readable medium having executable instructions stored thereon, such that, when the executable instructions are executed by the processor, the processor is further operative to derive a depth of the needle tip or the catheter tip relative to an imaging depth scale and present a numerical or graphical depth indicator on the composite overlay.
Aspect 39: The system of Aspect 24, wherein the one or more processors, and a computer-readable medium having executable instructions stored thereon, such that, when the executable instructions are executed by the processor, the processor is further operative to determine the position of the needle tip or the catheter tip based on a predetermined or measured distance between the magnetic element and the needle tip or the catheter shaft tip.
Aspect 40: The system of Aspect 24, wherein the one or more processors, and a computer-readable medium having executable instructions stored thereon, such that, when the executable instructions are executed by the processor, the processor is further operative to continuously update a three-dimensional position and orientation of the magnetic element by automatically calculating and/or analyzing vector measurements or information received from the plurality of magnetic sensors.
Aspect 41: The system of Aspect 24, wherein the plurality of electronic magnetic sensors comprise one or more of: one or more magnetometers, one or more Hall-effect sensors, one or more anisotropic magnetoresistive sensors, one or more tunneling magnetoresistance sensors, or one or more fluxgate sensors.
Aspect 42: The system of Aspect 24, wherein the magnetic element is a permanent magnet.
Aspect 43: The system of Aspect 24, wherein the magnetic element is an electromagnet configured to generate a controllable magnetic field in response to an applied current.
Aspect 44: The system of Aspect 24, wherein the medical imaging device is an ultrasound imaging device, wherein the one or more processors and the ultrasound imaging device are configured to perform both ultrasound image formation and magnetic tracking computation(s).
Aspect 45: The system of Aspect 24, wherein a rotational state of the magnetic element is represented on the composite overlay as a graphical or symbolic indicator of magnetic axis orientation or other procedural or event status indicator.
Aspect 46: The system of Aspect 24, wherein the one or more processors, and a computer-readable medium having executable instructions stored thereon, such that, when the executable instructions are executed by the processor, the processor is further operative to synchronize time stamps between magnetic sensor data and acquired image and/or image data frames to maintain or control positional accuracy and provide real time or substantially real time feedback during needle or catheter shaft advancement towards and/or away from the anatomical region.
Aspect 47: The system of Aspect 24, wherein the one or more processors, and a computer-readable medium having executable instructions stored thereon, such that, when the executable instructions are executed by the processor, the processor is further operative to record, store, or export, information related to needle or catheter shaft trajectory, position data, or rotation-based user inputs, as at least part of a procedural record.
Aspect 48: The system of Aspect 24, wherein the composite overlay includes a three-dimensional visualization of the needle or the catheter shaft trajectory relative to anatomical structures depicted in the image, the image data, and/or imaging geometry.
Aspect 49: The system of Aspect 24, wherein the one or more processors, and a computer-readable medium having executable instructions stored thereon, such that, when the executable instructions are executed by the processor, the processor is further operative to zero out or cancel out static magnetic fields based on information received by the one or more processors, the information generated by rotating the magnetic element and recording, measuring, or determining, changes to the magnetic axis.
Aspect 50: The system of Aspect 24, wherein the one or more processors, and a computer-readable medium having executable instructions stored thereon, such that, when the executable instructions are executed by the processor, the processor is further operative to: (a) receive data generated by rotating the magnetic element, (b) based on the data, record, measure, or determine changes to the orientation of the magnetic axis due to the rotating of the magnetic element, and (c) based on the data, the changes to the orientation of the magnetic axis, or both, zero out or cancel out static magnetic fields.
Aspect 51: The system of Aspect 24, wherein the one or more processors, and a computer-readable medium having executable instructions stored thereon, such that, when the executable instructions are executed by the processor, the processor is further operative to perform a magnetic calibration routine operative to measure and optionally cancel out one or more of, contributions of static or substantially static magnetic fields, static or substantially static magnetic fields, magnetic interference, one or more stray magnetic field, magnetic noise, or external magnetic disturbance, by detecting or recognizing changes in or to the magnetic field during a user-induced or manual rotation of the magnetic element.

Embodiments of the invention also include a computer-readable medium comprising one or more computer files comprising a set of computer-executable instructions for performing one or more of the calculations, steps, processes, and operations described and/or depicted herein. In exemplary embodiments, the files may be stored contiguously or non-contiguously on the computer-readable medium. Embodiments may include a computer program product comprising the computer files, either in the form of the computer-readable medium comprising the computer files and, optionally, made available to a consumer through packaging, or alternatively made available to a consumer through electronic distribution. As used in the context of this specification, a “computer-readable medium” is a non-transitory computer-readable medium and includes any kind of computer memory such as floppy disks, conventional hard disks, CD-ROM, Flash ROM, non-volatile ROM, electrically erasable programmable read-only memory (EEPROM), and RAM. In exemplary embodiments, the computer readable medium has a set of instructions stored thereon which, when executed by a processor, cause the processor to perform tasks, based on data stored in the electronic database or memory described herein. The processor may implement this process through any of the procedures discussed in this disclosure or through any equivalent procedure.

In other embodiments of the invention, files comprising the set of computer-executable instructions may be stored in computer-readable memory on a single computer or distributed across multiple computers. A skilled artisan will further appreciate, in light of this disclosure, how the invention can be implemented, in addition to software, using hardware or firmware. As such, as used herein, the operations of the invention can be implemented in a system comprising a combination of software, hardware, or firmware.

Embodiments of this disclosure include one or more computers or devices loaded with a set of the computer-executable instructions described herein. The computers or devices may be a general-purpose computer, a special-purpose computer, or other programmable data processing apparatus to produce a particular machine, such that the one or more computers or devices are instructed and configured to carry out the calculations, processes, steps, operations, algorithms, statistical methods, formulas, or computational routines of this disclosure. The computer or device performing the specified calculations, processes, steps, operations, algorithms, statistical methods, formulas, or computational routines of this disclosure may comprise at least one processing element such as a central processing unit (i.e., processor) and a form of computer-readable memory which may include random-access memory (RAM) or read-only memory (ROM). The computer-executable instructions can be embedded in computer hardware or stored in the computer-readable memory such that the computer or device may be directed to perform one or more of the calculations, steps, processes and operations depicted and/or described herein.

Additional embodiments of this disclosure comprise a computer system for carrying out the computer-implemented method of this disclosure. The computer system may comprise a processor for executing the computer-executable instructions, one or more electronic databases containing the data or information described herein, an input/output interface or user interface, and a set of instructions (e.g., software) for carrying out the method. The computer system can include a stand-alone computer, such as a desktop computer, a portable computer, such as a tablet, laptop, PDA, or smartphone, or a set of computers connected through a network including a client-server configuration and one or more database servers. The network may use any suitable network protocol, including IP, UDP, or ICMP, and may be any suitable wired or wireless network including any local area network, wide area network, Internet network, telecommunications network, Wi-Fi enabled network, or Bluetooth enabled network. In one embodiment, the computer system comprises a central computer connected to the internet that has the computer-executable instructions stored in memory that is operably connected to an internal electronic database. The central computer may perform the computer-implemented method based on input and commands received from remote computers through the internet. The central computer may effectively serve as a server and the remote computers may serve as client computers such that the server-client relationship is established, and the client computers issue queries or receive output from the server over a network.

The input/output interfaces may include a graphical user interface (GUI) which may be used in conjunction with the computer-executable code and electronic databases. The graphical user interface may allow a user to perform these tasks through the use of text fields, check boxes, pull-downs, command buttons, and the like. A skilled artisan will appreciate how such graphical features may be implemented for performing the tasks of this disclosure. The user interface may optionally be accessible through a computer connected to the internet. In one embodiment, the user interface is accessible by typing in an internet address through an industry standard web browser and logging into a web page. The user interface may then be operated through a remote computer (client computer) accessing the web page and transmitting queries or receiving output from a server through a network connection.

The present invention has been described with reference to particular embodiments having various features. In light of the disclosure provided above, it will be apparent to those skilled in the art that various modifications and variations can be made in the practice of the present invention without departing from the scope or spirit of the invention. One skilled in the art will recognize that the disclosed features may be used singularly, in any combination, or omitted based on the requirements and specifications of a given application or design. When an embodiment refers to “comprising” certain features, it is to be understood that the embodiments can alternatively “consist of” or “consist essentially of” any one or more of the features. Other embodiments of the invention will be apparent to those skilled in the art from consideration of the specification and practice of the invention.

It is noted that where a range of values is provided in this specification, each value between the upper and lower limits of that range is also specifically disclosed. The upper and lower limits of these smaller ranges may independently be included or excluded in the range as well. The singular forms “a,” “an,” and “the” include plural referents unless the context clearly dictates otherwise. It is intended that the specifications and examples be considered as exemplary in nature and that variations that do not depart from the essence of the invention fall within the scope of the invention. Further, all references cited in this disclosure are each individually incorporated by reference herein in their entireties and as such are intended to provide an efficient way of supplementing the enabling disclosure of this invention as well as provide background detailing the level of ordinary skill in the art.

As used herein, the term “about” refers to plus or minus 5 units (e.g., percentage) of the stated value.

Reference in the specification to “some embodiments”, “an embodiment”, “one embodiment” or “other embodiments” means that a particular feature, structure, or characteristic described in connection with the embodiments is included in at least some embodiments, but not necessarily all embodiments, of the inventions.

As used herein, the term “substantial” and “substantially” refers to what is easily recognizable to one of ordinary skill in the art.

It is to be understood that the phraseology and terminology employed herein is not to be construed as limiting and are for descriptive purpose only.

It is to be understood that while certain of the illustrations and figures may be close to the right scale, most of the illustrations and figures are not intended to be of the correct scale.

It is to be understood that the details set forth herein do not construe a limitation to an application of the invention.

Furthermore, it is to be understood that the invention can be carried out or practiced in various ways and that the invention can be implemented in embodiments other than the ones outlined in the description above.

1) A medical device comprising:
a base or hub connected directly or indirectly to a needle or a catheter shaft;
the needle or the catheter shaft comprising a distal tip, wherein the needle or the catheter shaft extends away from the base or the hub and towards the distal tip along a longitudinal axis; and
a magnetic element configured for rotational movement, such that as the magnetic element rotates, the orientation of the magnetic axis of the magnetic element changes relative to the longitudinal axis of the needle or the catheter shaft.

2) The medical device of claim 1, wherein the magnetic axis of the magnetic element is arbitrarily or randomly oriented relative to the longitudinal axis of the needle or the catheter shaft.

3) The medical device of claim 1, wherein the rotatable magnetic element is positioned in, on, or near, the hub, the base, the needle, or the catheter shaft, such that the orientation of the magnetic axis of the rotatable magnetic element is variable, undetermined, feedback-based, or determined in real time or substantially real time.

4) The medical device of claim 1, wherein the orientation of the magnetic axis of the magnetic element is transverse, orthogonal, or diagonal, to the longitudinal axis of the needle or the catheter shaft.

5) The medical device of claim 1, wherein the orientation of the magnetic axis of the magnetic element is directed across or to otherwise intersect or be non-coaxial with the longitudinal axis of the needle or the catheter shaft.

6) The medical device of claim 1, wherein the magnetic element comprises a ring-shaped body having an opening through which at least a portion of the needle or the catheter shaft extends.

7) The medical device of claim 1, wherein the magnetic element is a solid magnet, wherein the solid magnet is positioned in, on, or near, the needle or the catheter shaft, and wherein the longitudinal axis of the needle or the catheter shaft does not extend through the magnetic element.

8) The medical device of claim 1, wherein the magnetic element is magnetized in a diametric direction such that its magnetic axis is substantially perpendicular or substantially diagonal to the longitudinal axis of the needle or the catheter shaft.

9) The medical device of claim 1, wherein the orientation of the magnetic axis of the magnetic element is not aligned in a predetermined orientation relative to the hub, the base, the needle, or the catheter shaft, during assembly or manufacturing of part or all of the medical device, components of the medical device, the needle, the catheter shaft, or combinations thereof.

10) The medical device of claim 1, further comprising a rotational sleeve, bearing, or bushing, disposed between the magnetic element and the hub, the base, the needle, or the catheter shaft, configured to rotate or facilitate changing the orientation of the magnetic axis of the magnetic element relative to the longitudinal axis of the needle or the catheter shaft.

11) The medical device of claim 1, wherein the medical device comprises or is in operable communication with one or more processors and one or more sensors, wherein the one or more sensors are in operable communication with the one or more processors, wherein the magnetic element generates a magnetic field that is non-coaxial relative to the longitudinal axis of the needle or the catheter shaft, the magnetic field detectable by the one or more sensors, and wherein the one or more sensors, the one or more processors, the one or more sensors in operable communication with the one or more sensors, or combinations thereof, are operative to:
determine an orientation or a position of the medical device, the needle, the catheter shaft, the distal tip of the needle, or the distal tip of the catheter shaft;
detect an orientation of the magnetic axis of the magnetic element relative to the longitudinal axis of the needle or the catheter shaft;
continuously or periodically track the orientation of the magnetic axis of the magnetic element over time; or
combinations thereof.

12) The medical device of claim 1, wherein the magnetic element comprises a permanent magnet, wherein the magnetic element comprises an electromagnet configured to generate a magnetic field in response to an applied current, or wherein the magnetic element comprises both a permanent magnet and an electromagnet configured to generate a magnetic field in response to an applied current.

13) The medical device of claim 1, wherein the orientation of the magnetic axis of the magnetic element is completely or partially rotatable before or during a medical procedure, wherein the needle or the catheter shaft are inserted into a patient or a patient anatomy during the medical procedure.

14) A system for magnetic-based needle or catheter shaft tracking or guiding, comprising:
a base or a hub and a needle or a catheter shaft extending distally from the base or the hub along a longitudinal axis, the needle or the catheter shaft comprising a distal needle tip or distal catheter shaft tip;
a magnetic element having a magnetic axis that varies rotationally relative to the longitudinal axis of the needle or the catheter shaft, wherein the magnetic element is associated with or directly or indirectly connected to the base, the hub, the needle, or the catheter shaft; and
a medical imaging device comprising:
one or more imaging sensors configured to acquire image and/or image information of an anatomical region, wherein the one or more imaging sensors are in direct or indirect operable communication with one or more processors;
a plurality of electronic magnetic sensors in operative communication with the one or more processors, the plurality of electronic magnetic sensors (a) disposed on the medical imaging device, (b) disposed within the medical imaging device, or (c) configured to be placed or located on or near a patient or the anatomical region; and
the one or more processors, and a computer-readable medium having executable instructions stored thereon, such that, when the executable instructions are executed by the one or more processors, the one or more processors are operative to receive information from the one or more imaging sensors, information from the plurality of electronic magnetic sensors, or both, and use the received information to calculate, measure, or recognize, a magnetic field generated by the magnetic element in three dimensions to determine a location, an orientation, or both, of the needle or the catheter shaft.

15) The system of claim 14, wherein the one or more processors are further operative to perform both image formation and magnetic tracking computation(s), wherein the system further comprises a display configured to display a composite overlay of (a) the determined location, orientation, or both, of the needle or the catheter shaft, and (b) one or more images of the anatomical region based on the acquired image and/or image information of the anatomical region.

16) The system of claim 14, wherein the medical imaging device is configured for one or more of the following imaging modalities: ultrasound, fluoroscopy, x-ray, computed tomography, Magnetic Resonance Imaging (MRI), or optical.

17) The system of claim 15, wherein the composite overlay further includes at least one indicator representing the position, the orientation, a trajectory, or combinations thereof, of the needle, the catheter shaft, the distal needle tip, or the distal catheter shaft tip.

18) The system of claim 14, wherein the one or more processors, and a computer-readable medium having executable instructions stored thereon, such that, when the executable instructions are executed by the processor, the processor is further operative to receive information from the plurality of electronic magnetic sensors and, in response: output instructions; output information related to a position of the needle, the catheter shaft, the distal needle tip, or the distal catheter shaft tip; output information related to an orientation of the needle, the catheter shaft, the distal needle tip, the distal catheter shaft tip, or combinations thereof, to the system, a user of the system, or both.

19) The system of claim 15, wherein the one or more processors, and a computer-readable medium having executable instructions stored thereon, such that, when the executable instructions are executed by the processor, the processor is further operative to generate and display, in real time or substantially real time, (a) the composite overlay of the determined location, orientation, or both, of the needle or the catheter shaft, and (b) the one or more images of the anatomical region based on the acquired image and/or image information of the anatomical region.

20) The system of claim 15, wherein the composite overlay further includes one or more of, a depiction of the needle, a depiction of the catheter shaft, a depiction of the distal needle tip, a depiction of the distal catheter shaft tip, a predicted or suggested needle or catheter shaft trajectory, or a predicted or suggested distal needle tip or distal catheter shaft tip trajectory, which are optionally updated on the composite overlay in real time or substantially real time.

21) The system of claim 14, wherein the plurality of electronic magnetic sensors surround or substantially surround a portion of the longitudinal axis of the needle or the catheter shaft, and wherein the plurality of electronic magnetic sensors and the portion of the longitudinal axis of the needle or the catheter shaft are optionally located within the medical imaging device or optionally directly or indirectly connected to the medical imaging device.

22) The system of claim 14, wherein the medical imaging device is an ultrasound imaging device, wherein the ultrasound imaging device includes a probe body, and wherein the probe body includes two ultrasound arrays housed within the probe body, and wherein the longitudinal axis of the needle or the catheter shaft is positioned between the two ultrasound arrays.

23) The system of claim 14, wherein the one or more processors, and a computer-readable medium having executable instructions stored thereon, such that, when the executable instructions are executed by the processor, the processor is further operative to cancel out one or more of, contributions of static or substantially static magnetic fields, static or substantially static magnetic fields, magnetic interference, one or more stray magnetic field, magnetic noise, or external magnetic disturbance, by detecting or recognizing changes in or to the magnetic field.

24) The system of claim 14, wherein a user-manipulated or manual rotation of the magnetic element relative to a sensing geometry of the plurality of electronic magnetic sensors is detected or recognized by the one or more processors as a user-interface command input, and wherein the user-manipulated or manual rotation of the magnetic element through a predefined angular increment is configured to cause the one or more processors to execute a predefined function.

25) The system of claim 24, wherein the predefined function comprises one or more of:
(a) initiating needle or catheter shaft tracking mode;
(b) marking a start and/or an end of a procedure phase or event during which the needle or the catheter shaft and/or the system are used;
(c) recording a time interval between procedure phases or events; or
(d) toggling a calibration of a displayed composite overlay state.

26) The system of claim 15, wherein the one or more processors, and a computer-readable medium having executable instructions stored thereon, such that, when the executable instructions are executed by the processor, the processor is further operative to derive a depth of the distal needle tip or the distal catheter shaft tip relative to an imaging depth scale and present a numerical or graphical depth indicator on the composite overlay.

27) The system of claim 14, wherein the one or more processors, and a computer-readable medium having executable instructions stored thereon, such that, when the executable instructions are executed by the processor, the processor is further operative to determine the position of the distal needle tip or the distal catheter tip based on a predetermined or measured distance between the magnetic element and the distal needle tip or the distal catheter shaft tip.

28) The system of claim 14, wherein the one or more processors, and a computer-readable medium having executable instructions stored thereon, such that, when the executable instructions are executed by the processor, the processor is further operative to continuously update a three-dimensional position and orientation of the magnetic element by automatically calculating and/or analyzing vector measurements or the information received from the plurality of magnetic sensors.

29) The system of claim 14, wherein the magnetic element comprises a permanent magnet, wherein the magnetic element comprises an electromagnet configured to generate a magnetic field in response to an applied current, or wherein the magnetic element comprises both a permanent magnet and an electromagnet configured to generate a magnetic field in response to an applied current.

30) The system of claim 14, wherein the one or more processors, and a computer-readable medium having executable instructions stored thereon, such that, when the executable instructions are executed by the processor, the processor is further operative to synchronize time stamps between received information from the plurality of electronic magnetic sensors, the acquired image and/or image information from the one or more imaging sensors, or both, to maintain or control positional accuracy and provide real time or substantially real time feedback during needle or catheter shaft advancement towards and/or away from the anatomical region.
